(19) United States
(12) Patent Application Publication
(10) Pub. No.: US 2022/0095963 A1
(43) Pub. Date: Mar. 31, 2022

(54) IMPLANTABLE DEVICE INCLUDING A FLEXIBLE BIOCHEMICAL SENSOR AND METHOD OF MANUFACTURE THEREOF

(71) Applicant: The Board of Trustees of the Leland Stanford Junior University, Stanford, CA (US)

(72) Inventors: Zhenan Bao, Stanford, CA (US); Jinxing Li, Stanford, CA (US); Yuxin Liu, Stanford, CA (US)

(73) Assignee: The Board of Trustees of the Leland Stanford Junior University, Stanford, CA (US)

(21) Appl. No.: 17/489,594

(22) Filed: Sep. 29, 2021

Related U.S. Application Data
(60) Provisional application No. 63/085,720, filed on Sep. 30, 2020.

(51) Int. Cl. A61B 5/1473; A61B 5/145; A61B 5/1459; A61N 5/06; A61N 1/05; B23K 26/364

(52) U.S. Cl. CPC A61B 5/1473; A61B 5/14546; A61B 5/1459; A61N 5/0601; A61N 2005/063; A61N 1/0526; B23K 26/364; A61B 2562/125; A61N 1/0509

(57) ABSTRACT

Present implementations can include a method of forming an implantable biochemical sensor, by coating a first substrate with a first solution, etching, by a laser, the first substrate to form one or more electrodes in the first substrate, coating a first face of the electrodes with an elastomer solution, coating a second face of the electrodes with the elastomer solution, solidifying the elastomer coating into an elastomer shell at least partially surrounding the electrodes, and removing at least a portion of the elastomer shell to form implantable electrodes.

### CROSS-REFERENCE TO RELATED PATENT APPLICATIONS

This application claims priority to U.S. Provisional Patent Application Ser. No. 63/085,720, entitled “LASER INDUCED CONDUCTIVE MATERIALS FOR STRETCHABLE ELECTRONICS,” filed Sep. 30, 2020, the contents of all such applications being hereby incorporated by reference in its entirety and for all purposes as if completely and fully set forth herein.

### TECHNICAL FIELD

The present implementations relate generally to medical devices, and more particularly to an implantable device including a flexible biochemical sensor.

### BACKGROUND

Soft tissues and organs in the human body are highly active biochemical systems that include biomolecules, such as neurotransmitters and hormones, precisely controlling various biological processes. Monitoring the dynamics of neurotransmitters is essential to understanding the communication between neurons and their targets, and to develop therapeutic neuromodulatory strategies. In the central nervous system (CNS), monoamines, including dopamine (DA) and serotonin (5-HT), are involved in the regulation of cognitive processes such as emotion, arousal, and memory. Dysregulated monoamine signaling is a common feature of many psychiatric and neurological disorders, including addiction, major depressive disorder, and Parkinson's disease. Outside the CNS, 5-HT in the gastrointestinal (GI) system accounts for 95% of the body's 5-HT and closely regulates gut function and microbiota, serving as an important component of the gut-brain communication system. Therefore, monitoring the dynamics of monoamines in both the CNS and the GI system is desired. However, conventional system lack sufficient responsiveness to current presence of biochemicals including dopamine and serotonin in the CNS and the GI system.

### SUMMARY

Present implementations are directed to sensor devices implantable into diverse in vivo biological environments, and operable to sense current conditions of multiple biochemicals and the like within those in vivo biological environments. Present implementations are advantageously compatible with soft tissues of multiple distinct in vivo environments, possess structures advantageously compatible with particular aspects of in vivo environments to which they are implanted and implantable. For example, sensor devices in accordance with present implementations are implantable in or on, for example, living brain tissue and gastrointestinal organ structures. Sensor devices as discussed herein can include flexible electrical and electronic structures, and can include flexible housings and encapsulations to advantageously allow implantation into in vivo environments and affixation with in vivo organs without interfering with biological activity in the implantation area, organs to which the sensor device is affixed, or overall movement and activity of a biological organism. Sensor devices can be deformable, bendable, and stretchable, for example, and can move in accordance with expansion, contraction, compression, deformation, or the like, of an organ or organism surrounding the point of implantation.

Implantable sensor devices in accordance with present implementations can be electrically responsive to the presence of multiple biochemicals and the like, both common to and particular to various in vivo environments. The sensor devices can also be concurrently responsive to multiple biochemicals to report out the presence and amount of multiple biochemicals at an in vivo location of an organism. As one example, a sensor device can include multiple electrodes implanted at a particular in vivo location, where each electrode is electrically responsive to a distinct biochemical. The sensor devices can also be responsive to the same biochemical and can amplify an electrical response of the sensor device to presence or amount of the biochemical. It is to be understood that the sensor devices in accordance with present implementations are not limited to in vivo environments, organs, implantation sites, or the like, particularly described herein. The sensor devices can be implantable at any location, organ, site, or the like, to be responsive to one or more of at least the biochemicals discussed herein.

Present implementations can include a tissue-mimicking, stretchable neurochemical biointerface prepared by laser-patterning a metal-complexed polyimide into an interconnected graphene/nanoparticle network embedded in an elastomer. Sensor devices in accordance with present implementations can allow real-time, persistent, chronic, multichannel, and multiplexed in vivo sensing in real-time. The real-time sensing can include measuring monoamines in the brain in real-time, and measuring serotonin dynamics in the gut in real-time without undesired stimulations and perturbing peristaltic movements. The described elastic and conformable biosensing interface can identify effect of neurotransmitters on gut microbes, detect brain-gut communication, and is applicable to biomolecular sensing in other soft organs across the body. Thus, a technological solution for an implantable device including a flexible biochemical sensor is provided.

Present implementations can include a method of forming an implantable biochemical sensor, by coating a first substrate with a first solution, etching, by a laser, the first substrate to form one or more electrodes in the first substrate, coating a first face of the electrodes with an elastomer solution, coating a second face of the electrodes with the elastomer solution, solidifying the elastomer coating into an elastomer shell at least partially surrounding the electrodes, and removing at least a portion of the elastomer shell to form implantable electrodes.

Present implementations can include a method where the removing further includes removing the portion of the elastomer shell to form implantable electrode strings.

Present implementations can include a method where the electrode strings each have a width less than 100 μm.

Present implementations can include a method where the removing further includes removing the portion of the elastomer shell to form an implantable electrode probe.

Present implementations can include a method of forming a shuttle layer on the implantable electrodes.

Present implementations can include a method of dissolving at least a portion of the shuttle layer by contact with an in vivo neurochemical environment.

Present implementations can include a method of dissolving at least a portion of the shuttle layer by contact with an in vivo gastrointestinal environment.

Present implementations can include a method of applying a biochemical sensor layer to corresponding implantable ends of the electrodes.

Present implementations can include a method where the first solution includes one or more of polyamic acid and metalloporhyrin.

Present implementations can include a method where the electrodes include a graphene network.

Present implementations can include a method of transforming, by the laser, the first substrate and the first coating into the graphene network. Present implementations can include a method of generating, by the laser, nanoparticles within the graphene network.

Present implementations can include an implantable biochemical sensor device, with a first biochemical sensor including a first portion of a graphene network, and having an elongated structure with a first input end and a first sensor end distal to the first input end, and a second biochemical sensor including a second portion of the graphene network, and having the elongated structure with a second input end and a second sensor end distal to the second input end.

Present implementations can include a device where the first sensor end and the second sensor end each include implantable electrode strings each having a width less than less than 100 μm.

Present implementations can include a device where the first sensor end and the second sensor end are electrically responsive to one or more of dopamine, serotonin, norepinephrine, and epinephrine.

Present implementations can include a device with an elastomer coating at least partially surrounding the first biochemical sensor and the second biochemical sensor.

Present implementations can include a device with an elastomer coating individually surrounding each of the first sensor end and the second sensor end.

Present implementations can include a device with an optical fiber operatively coupled with at least one of the first biochemical sensor and the second biochemical sensor, the optical fiber operable to perform optogenetic stimulation within an in vivo environment.

Present implementations can include a device where the graphene network is operable to transmit an electrical stimulation signal and receive an electrical signal from an in vivo environment.

Present implementations can include a device where at least a portion of the graphene includes at least one of a biochemical receptor and a chemical receptor.

### BRIEF DESCRIPTION OF THE DRAWINGS

These and other aspects and features of the present implementations will become apparent to those ordinarily skilled in the art upon review of the following description of specific implementations in conjunction with the accompanying figures, wherein:

FIG. 1A illustrates a first implantable sensor device in a plan view, in accordance with present implementations.

FIG. 1B illustrates a first implantable sensor device in a cross-sectional view, further to the example sensor device of FIG. 1A.

FIG. 1C illustrates a first implantable sensor device in an implantation state and in a plan view, further to the example sensor device of FIG. 1A.

FIG. 2A illustrates a second implantable sensor device in a plan view, in accordance with present implementations.

FIG. 2B illustrates a second implantable sensor device in a cross-sectional view, further to the example sensor device of FIG. 2A.

FIG. 2C illustrates a first implantable sensor device in an implantation state and in a plan view, further to the example sensor device of FIG. 2A.

FIG. 3 illustrates a first example operating response of an implantable sensor device in accordance with present implementations.

FIG. 4 illustrates a second example operating response of an implantable sensor device in accordance with present implementations.

FIG. 5 illustrates a third example operating response of an implantable sensor device in accordance with present implementations.

FIG. 6 illustrates a method of manufacturing an implantable device including a flexible biochemical sensor, in accordance with present implementations.

FIG. 7 illustrates a method of manufacturing an implantable device including a flexible biochemical sensor, further to the example method of FIG. 6.

FIG. 8 illustrates a method of manufacturing a brain-implantable or gastrointestinal-implantable device including a flexible biochemical sensor, further to the example method of FIG. 7.

FIG. 9 illustrates a method of manufacturing an implantable device including a flexible biochemical sensor, further to the example method of FIG. 8.

### DETAILED DESCRIPTION

The present implementations will now be described in detail with reference to the drawings, which are provided as illustrative examples of the implementations so as to enable those skilled in the art to practice the implementations and alternatives apparent to those skilled in the art. Notably, the figures and examples below are not meant to limit the scope of the present implementations to a single implementation, but other implementations are possible by way of interchange of some or all of the described or illustrated elements. Moreover, where certain elements of the present implementations can be partially or fully implemented using known components, only those portions of such known components that are necessary for an understanding of the present implementations will be described, and detailed descriptions of other portions of such known components will be omitted so as not to obscure the present implementations. Implementations described as being implemented in software should not be limited thereto, but can include implementations implemented in hardware, or combinations of software and hardware, and vice-versa, as will be apparent to those skilled in the art, unless otherwise specified herein. In the present specification, an implementation showing a singular component should not be considered limiting; rather, the present disclosure is intended to encompass other implementations including a plurality of the same component, and vice-versa, unless explicitly stated otherwise herein. Moreover, applicants do not intend for any term in the specification or claims to be ascribed an uncommon or special meaning unless explicitly set forth as such. Further, the present implementations encompass present and future known equivalents to the known components referred to herein by way of illustration.

Neurotransmitters play essential roles in regulating neural circuit dynamics both in the central nervous system as well as at the peripheral, including the gastrointestinal tract. Real-time monitoring of these biological structures can offer critical information for understanding neural function and diagnosing disease. However, probing soft, complex, and actively moving organs increases the complexity of effectively monitoring these biological structures. Voltammetry neurotransmitter can be advantageously achieved with soft structures, for both comfort and durability within in vivo environments. As one example, silica encapsulated carbon fiber electrodes, which are rigid and brittle, possess limited tunability of sensing functions. These rigid probes can lead to early device failure and severe inflammatory response. Rigid implantable sensor devices can cause device failure and severe inflammatory response as the brain is undergoing constant motion and deformation due to the cardiorespiratory cycles and body movements. Similarly, the GI tract is made of a series of soft, long, and twisting organs. Further, these organs move in a variety of motility patterns, including peristaltic and non-peristaltic patterns, and including many mechanoreceptors. Performing high-fidelity electrical or optical measurements of 5-HT dynamics in an actively moving GI tract has been a long-standing challenge.

Present implementations can include a soft bioelectronic interface operable to monitor the dynamics of the monoamine neurotransmitter, including DA and 5-HT levels, in both the brain and the gut of living organisms, including humans and animals. With tissue-like mechanical property, example sensors can seamlessly interface with actively moving organs. These sensors are compatible with traditional medical inspection devices including, for example, endoscopy for noninvasive biomolecular monitoring. Thus, implantable sensor devices in accordance with present implementations can achieve chronically stable and multiplexed neurochemical sensing in the CNS. In the GI tract, high stretchability and softness of example sensor devices advantageously achieves conformability with intestinal tissue without disturbing the peristaltic movement and without inducing undesired stimulation. Unique elastic features of elastomer structures and electrode structures of example sensor devices are advantageously suitable for simultaneous monitoring of neurotransmitter signaling from both the central and peripheral nervous systems, and can advantageously monitor the dynamics of gut chemistry and its interplay with microbes. As discussed above, implantable sensor devices in accordance with present implementations can also simultaneously sense multiple biomolecules at multiple locations.

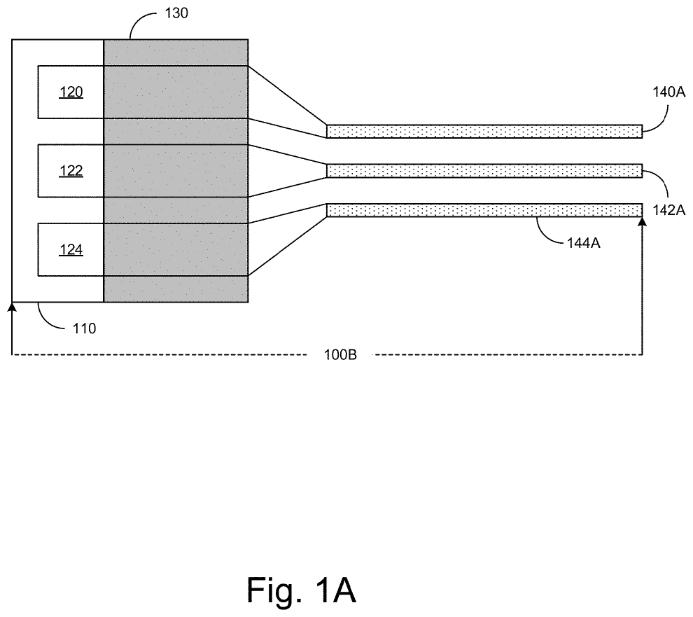

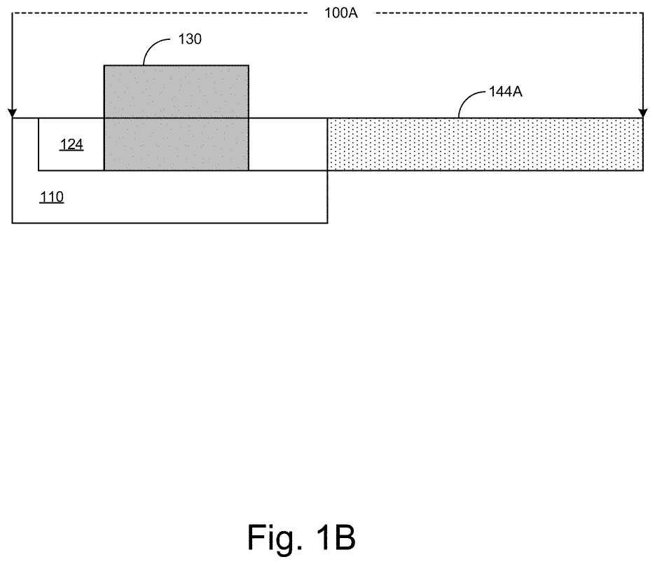

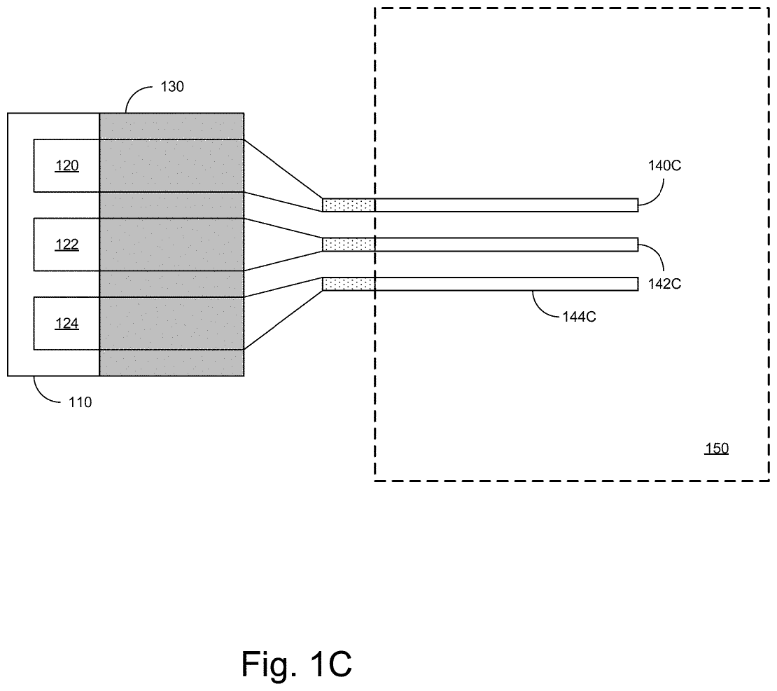

FIGS. 1A-C are directed to a sensor device 100A-C implantable or implanted within living brain tissue of an in vivo environment or the like. The sensor device 100A-C can include a patterned graphene-elastomer composite housing that can be easily stretched, twisted, and knotted. As one example, the sensor device 100A-C can include, but is not limited to, a stretched 3-channel sensor device, where each channel can separately and independently sense brain neurotransmitter activity and generate separate and independent electrical responses thereto. It is to be understood that sensor devices in accordance with present implementations are not limited to the number of electrodes, electrode strings, or sensor probes illustrated herein. The sensor electrodes can be variously combined with each other with varying functions with respect to at least chemical and mechanical sensing properties, and can be distributed and embedded inside one or more elastomers. As one example, strain, temperature or pressure sensing electrodes can be completely enclosed within an elastomer, and chemical sensing electrodes and electrical stimulation electrodes can be at least partially exposed to be directly contactable with an in vivo biological object. Such a string can be combined with an optical fiber or the like to effect a wired electrical communication channel from an in vivo environment to an external computing or communication device or system, for example. Such a string can also be used as a soft and stretchable optical fiber to deliver light to the body for optical therapy or to deliver the light to the brain for optogenetic stimulation. Such a string can also be used as a soft and stretchable optical sensor to be implanted in the body to illuminate the target and to read the optical signal for fluorescence or other optical imaging and sensing. An elastomer can be a medium for optical signal transmission through the sensor device, and can be transparent or translucent to facilitate optical signal transmission therethrough.

FIG. 1A illustrates a first implantable sensor device in a plan view, in accordance with present implementations. As illustrated by way of example in FIG. 1A, an implantable sensor device in a plan view 100A can include a sensor substrate 110, sensor electrodes 120, 122 and 124, an encapsulation layer 130, and implantable sensor strings 140A, 142A and 144A. The sensor device 100A can include one or more electrodes and implantable sensor strings, but is not limited to the number of electrodes and sensor strings illustrated and described herein. The number of electrodes and sensor string can be higher or lower than the number illustrated and described herein.

The sensor substrate 110 can include at least one nondestructively deformable material disposed at least partially surrounding one or more of the sensor electrodes 120, 122 and 124 or the sensor strings 140A, 142A, and 144A. Nondestructive deformation can include a deformation of an object in a lateral, axial, angular direction, for example, from which the object can return to its original shape. The sensor substrate 110 can include an elastomer matrix formed from an elastomeric solution or the like. The elastomer matrix can include at least one styrenic block copolymer, silicon elastomer, fluoroether elastomer, fluoropolymer elastomer, polyurethane or other elastomers or any combination thereof. As one example, the sensor substrate 110 can include polystyrene-block-poly(ethylene-ran-butylene)-block-polystyrene (SEBS). The portion of the sensor substrate 110 in contact with the encapsulation layer 130 can be of an arbitrary length in a direction extending from the sensor electrodes 120, 122 and 124 toward the sensor string 140A, 142A and 144A, and can form an extended portion, ribbon, insulated wire, for example, coupling the sensor strings 140A, 142A, and 144A with the sensor electrodes 120, 122 and 124.

The sensor electrodes 120, 122 and 124 can include destructively deformable material arranged in electrically conductive tracks, and can include a material electrically responsive to one or more biochemicals. The sensor electrodes 120, 122 and 124 can form a soft and stretchable graphene-based biosensing neural interface to enable real-time simultaneous monitoring of monoamine dynamics in at least both brain and gastrointestinal tissues. The portion of the sensor electrodes 120, 122 and 124 in contact with the encapsulation layer 130 can be of an arbitrary length in a direction extending from the toward the sensor string 140A, 142A and 144A, and can form an extended portion, ribbon, insulated wire, for example, coupling the sensor strings 140A, 142A, and 144A with the sensor electrodes 120, 122 and 124. The sensor electrodes 120, 122 and 124 can further be contacted with one or more electrical components to obtain electrical responses from the sensor strings 140A, 142A, and 144A, and to generate one or more biochemical response outputs based on the electrical response from the sensor strings 140A, 142A, and 144A. Biochemical response outputs can include, for example, quantitative values in the form or digital or analog signals indicating one or more or presence and amount of one or more biochemicals at the in vivo site proximate to the sensor strings 140A, 142A, and 144A.

The encapsulation layer 130 can include at least one nondestructively deformable material disposed at least partially surrounding one or more of the sensor electrodes 120, 122 and 124 or the sensor strings 140A, 142A, and 144A. As one example, the encapsulation layer 130 can include SEBS, similarly to the sensor substrate 110. The encapsulation layer 130 can be formed covering one or more faces of the sensor electrodes 120, 122 and 124 opposite faces covered by the sensor substrate 110. Thus, the encapsulation layer 130 and the sensor substrate 110 can sandwich the sensor electrodes 120, 122 and 124. The encapsulation layer 130 can leave at least a portion of sensor electrodes 120, 122 and 124 exposed to provide external surfaces of the sensor electrodes 120, 122 and 124 that are electrically contactable with external, wires, electronics, and the like, to capture and process the electrical responses of the sensor strings 140A, 142A and 144A transmitted to the sensor electrodes 120, 122 and 124 by physical contact therebetween. The sensor substrate 110 can form a buffer region between electrode 120 and sensor string 140A, 142A, and 144A, to advantageously isolate the sensor electrodes 120, 122 and 124 from any in vivo environment proximate to the sensor string 140A, 142A and 144A.

The implantable sensor strings 140A, 142A and 144A can include destructively deformable material arranged in electrically conductive tracks, lines, or traces, for example, and can include a material electrically responsive to one or more biochemicals. The implantable sensor strings 140A, 142A and 144A can be soft, elastic, thin and long to minimize damage to brain tissue and can seamlessly interface with CNS tissue by direct contact with those tissues or positioning proximate to the tissues. As one example, the implantable sensor strings 140A, 142A and 144A can include graphene material disposed on the sensor substrate 100 or integrated with the sensor substrate 110. Graphene materials can advantageously demonstrate biocompatibility with at least CNS and GI tissue and in vivo environments. Further graphene material can demonstrate advantageous electrochemical properties including a super-capacitive response during fast-scan cyclic voltammetry (FSCV), and known catalytic activity towards amine oxidation. From a mechanical perspective, graphene can also advantageously demonstrate nondestructive mechanical deformation and restoration in bending, stretching and twisting due to its atomic-level thickness. In some implementations, graphene monolayer can demonstrate nondestructive deformation when subjected to less than 5% strain. Embedding laser-induced graphene nanofiber networks with transition metal nanoparticles decorated on the surface, into a elastomer matrix can increase nondestructive deformation induced by strain significantly, and can achieve high levels of softness and stretchability while preserving the unique electrochemical properties of the nanomaterials. The graphene nanofiber can have a thickness of under 100 μm, and approximately 50-80 μm. The graphene nanofiber can include at least one of a biochemical receptor and a chemical receptor, located on or within at least a portion thereof. As one example, the graphene nanofiber can be treated with a material responsive to a particular chemical or biochemical at least as discussed herein. The graphene nanofiber can also transmit an electrical stimulation signal to an in vivo environment, and can receive an electrical signal from the in vivo environment. The received electrical signal can include, for example, electrical signals associated with neural activity, gastrointestinal activity, muscle activity, or the like.

The implantable sensor strings 140A, 142A and 144A can include a shuttle layer at least partially surrounding ends of each of the implantable sensor strings 140A, 142A and 144A. As one example, the shuttle layer can extend to completely cover each of the implantable sensor strings 140A, 142A and 144A individually, and can provide rigidity to the sensor strings. The rigidity of the shuttle layer can advantageously assist in implantation of the implantable sensor strings 140A, 142A and 144A within the in vivo environment. Specifically, the rigidity of the shuttle layer can increase accuracy and ease of an initial placement of the implantable sensor strings 140A, 142A and 144A at a particular location within the in vivo environment and can assist in penetration of or entry into the in vivo environment without deformation.

FIG. 1B illustrates a first implantable sensor device in a cross-sectional view, further to the example sensor device of FIG. 1A. As illustrated by way of example in FIG. 1B, an implantable sensor device in a cross-sectional view 100B can include the sensor substrate 110, the sensor electrode 124, the encapsulation layer 130, and the implantable sensor strings 144A.

FIG. 1C illustrates a first implantable sensor device in an implantation state and in a plan view, further to the example sensor device of FIG. 1A. As illustrated by way of example in FIG. 1C, an implantable sensor device 100C in an implantation state and in a plan view can include the sensor substrate 110, the sensor electrodes 120, 122 and 124, the encapsulation layer 130, and implanted sensor strings 140C, 142C and 144C in contact with a biological object 150. An optical fiber can be operatively coupled with electrode of the sensor device or inserted alongside the sensor device into contact with a particular portion of the brain within the in vivo environment. The optical fiber can be operable to perform optogenetic stimulation within the in vivo environment, and can stimulate a particular brain region or structure, for example.

The implanted sensor strings 140C, 142C and 144C can be implanted into an in vivo environment including the biological object 150. Each of the implanted sensor strings 140C, 142C and 144C can be individually implanted by insertion into the in vivo environment. Insertion can be compatible with or subsequent to, for example, an opening through a minimally invasive surgical procedure or keyhole surgery. The biological object 150 can include a neurochemical environment of a living organism. The biological object 150 can be brain tissue, a living brain, an internal cranial cavity, and the like, for example. The implanted sensor strings 140C, 142C and 144C can be in contact with various portions of the brain within the biological organism, including neurotransmitter regions, particular lobes or cognitive regions of the brain, and particular groups of neurons within the brain. The shuttle layer surrounding the implanted sensor strings 140C, 142C and 144C can dissolve after a predetermined period of time in contact with the in vivo environment, to return the implanted sensor strings 140C, 142C and 144C to their flexible state after implantation.

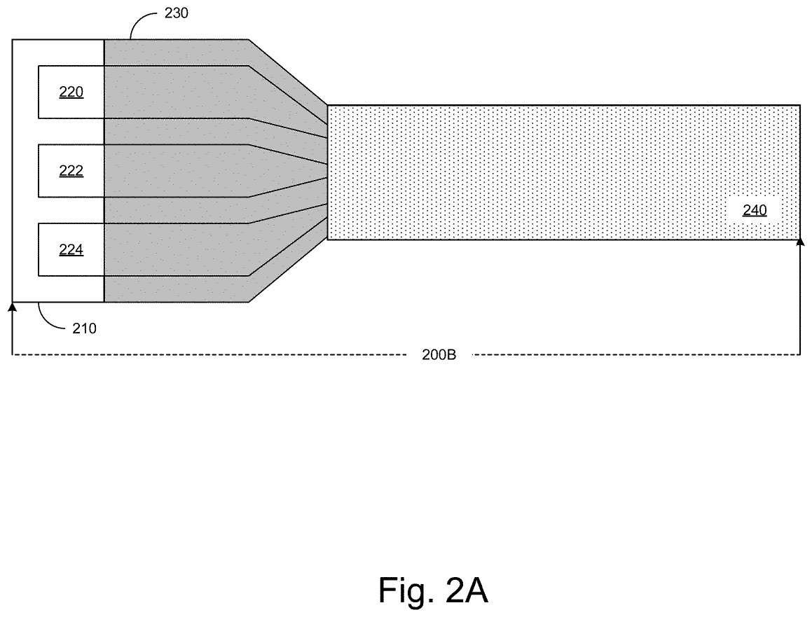

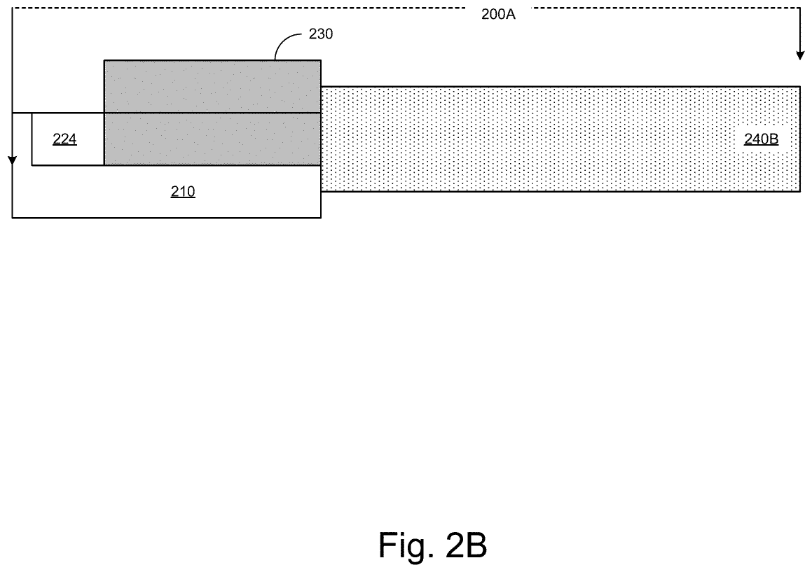

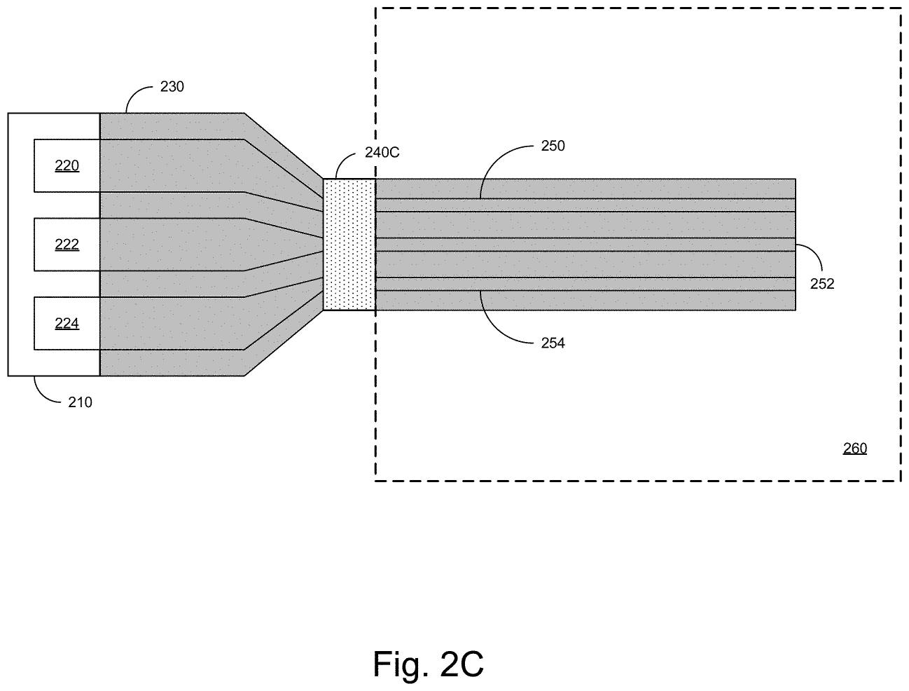

FIGS. 2A-C are directed to a sensor device 200A-C implantable or implanted within living gastrointestinal tissue of an in vivo environment or the like. The sensor device 200A-C can include a patterned graphene-elastomer composite housing that can be easily stretched, twisted, and knotted similarly to that of sensor device 100A-C. As one example, the sensor device 200A-C can include, but is not limited to, a stretched 3-channel sensor device, where each channel can separately and independently sense brain neurotransmitter activity and generate separate and independent electrical responses thereto.

FIG. 2A illustrates a second implantable sensor device in a plan view, in accordance with present implementations. As illustrated by way of example in FIG. 2A, an implantable sensor device 200A in a plan view can include a sensor substrate 210, sensor electrodes 220, 222 and 224, an encapsulation layer 230, and an implantable sensor probe 240. The sensor device 200A can include one or more electrodes and implantable sensor strings, but is not limited to the number of electrodes illustrated and described herein. The number of electrodes can be higher or lower than the number illustrated and described herein.

The sensor substrate 210 can include at least one nondestructively deformable material disposed at least partially surrounding one or more of the sensor electrodes 120, 122 and 124 or the implantable sensor probe 240. The sensor substrate 210 can correspond at least partially in one or more of structure and operation to the sensor substrate 110. In addition, the sensor substrate can extend to partially cover one or more sensor leads within the implantable sensor probe 240 area of the sensor device 200A. The sensor electrodes 220, 222 and 224 can include destructively deformable material arranged in electrically conductive tracks, and can include a material electrically responsive to one or more biochemicals. The sensor electrodes 220, 222 and 224 can correspond at least partially in one or more of structure and operation to the sensor electrodes 120, 122 and 124.

The encapsulation layer 230 can include at least one nondestructively deformable material disposed at least partially surrounding one or more of the sensor electrodes 220, 222 and 224 or the sensor leads within the implantable sensor probe 240 area of the sensor device 200A. The encapsulation layer 230 can extend in a direction from the electrodes 220, 222 and 224 to the implantable sensor probe 240 area. The encapsulation layer 230 can terminate in contact with the implantable sensor probe 240 to provide a seal to exclude any potential contact with an in vivo environment proximate to the implantable sensor probe 240 area and any ambient environment surrounding the sensor device 200A. The encapsulation layer 230 can correspond at least partially in one or more of structure and operation to the encapsulation layer 130.

The implantable sensor probe 240 can include destructively deformable material at least partially enclosing one or more sensor leads. The sensor leads can include electrically conductive tracks, lines, or traces, for example, and can include a material electrically responsive to one or more biochemicals, correspondingly to the graphene of the sensor strings 140A, 142A and 144A. The implantable sensor probe 240 can be soft, elastic, thin and long to minimize damage to GI tissue and can seamlessly interface with GI tissue by direct contact with those tissues or positioning proximate to the tissues. As one example, the implantable sensor probe 240 can be placed adjacent to, over, or wrapped around a particular biological structure, including tubes or the like of the in vivo GI system. The implantable sensor probe 240 for the gut is on a single thin and elastic film to facilitate device operation and measurements over a much longer distance and a larger degree of movement than with respect to the sensor strings 140A, 142A and 144A optimized for the neurochemical environment. Thus, the implantable sensor probe 240 can accommodate strain and nondestructive deformation caused by the higher level of movement in the GI system than in the CNS system. The implantable sensor probe 240 can include a shuttle layer at least partially surrounding implantable sensor probe 240. As one example, the shuttle layer can extend to completely cover the implantable sensor probe 240 in a single coating, and can provide rigidity to implantable sensor probe 240, similarly to the sensor strings 140A, 142A and 144A.

FIG. 2B illustrates a second implantable sensor device in a cross-sectional view, further to the example sensor device of FIG. 2A. As illustrated by way of example in FIG. 2B, an implantable sensor device 200B in a cross-sectional view can include the sensor substrate 210, the sensor electrode 224, the encapsulation layer 230, and the implantable sensor probe 240.

FIG. 2C illustrates a first implantable sensor device in an implantation state and in a plan view, further to the example sensor device of FIG. 2A. As illustrated by way of example in FIG. 2C, an implantable sensor device 200C in an implantation state and in a plan view can include the sensor substrate 210, the sensor electrodes 220, 222 and 224, the encapsulation layer 230, the implanted sensor probe 240C, and sensor leads 250, 252 and 254 in contact with a biological object 260. An optical fiber can be operatively coupled with electrode of the sensor device or inserted alongside the sensor device into contact with a particular portion of the gastrointestinal system within the in vivo environment. The optical fiber can be operable to perform optogenetic stimulation within the in vivo environment, and can stimulate a particular gastrointestinal region or structure, for example.

The implanted sensor probe 240C can be implanted into an in vivo environment including the biological object 150. The implanted sensor probe 240C can be implanted by insertion into the in vivo environment. Insertion can be compatible with or subsequent to, for example, an opening through a minimally invasive surgical procedure or keyhole surgery. The shuttle layer surrounding the implanted sensor probe 240C can dissolve after a predetermined period of time in contact with the in vivo environment or the biological object 260, to return the implanted sensor probe 240C to its flexible state after implantation.

The sensor leads 250, 252 and 254 can include destructively deformable material arranged in electrically conductive tracks, lines, or traces, for example, and can include a material electrically responsive to one or more biochemicals. As one example, the sensor leads 250, 252 and 254 can be at least partially exposed to the in vivo environment and the biological object 260 by the dissolving of the shuttle layer surrounding the sensor leads 250, 252 and 254. The sensor leads 250, 252 and 254 can correspond at least partially in one or more of structure and operation to the sensor strings 140A, 142A, 144A, 140C, 142C and 144C. As one example, the sensor leads 250, 252 and 254 can detect 5-HT. 5-HT can serve as a biomarker for gut inflammation, and the sensor leads 250, 252 and 254 can be used for IBD diagnosis and studying the gut chemistry in living organisms including humans. The flexibility of the sensor device 200A-C allows it to be wrapped on an endoscope to enable rapid mapping of colon 5-HT by navigating the endoscope working channel while performing electrochemical sensing by the sensor device 200C.

The biological object 260 can include a GI environment of a living organism. The biological object 260 can be intestinal tissue, a living stomach, an internal abdominal cavity, and the like, for example. The implanted sensor probe 240C can be in contact with various portions of the GI system within the biological organism, including intestinal regions, and outer surfaces of stomach or intestinal organs, for example. As one example, real-time 5-HT recording using the sensor device 200C by chronoamperometry can result in an electrical response indicating a stable 5-HT concentration during the peristalsis motion of the colon.

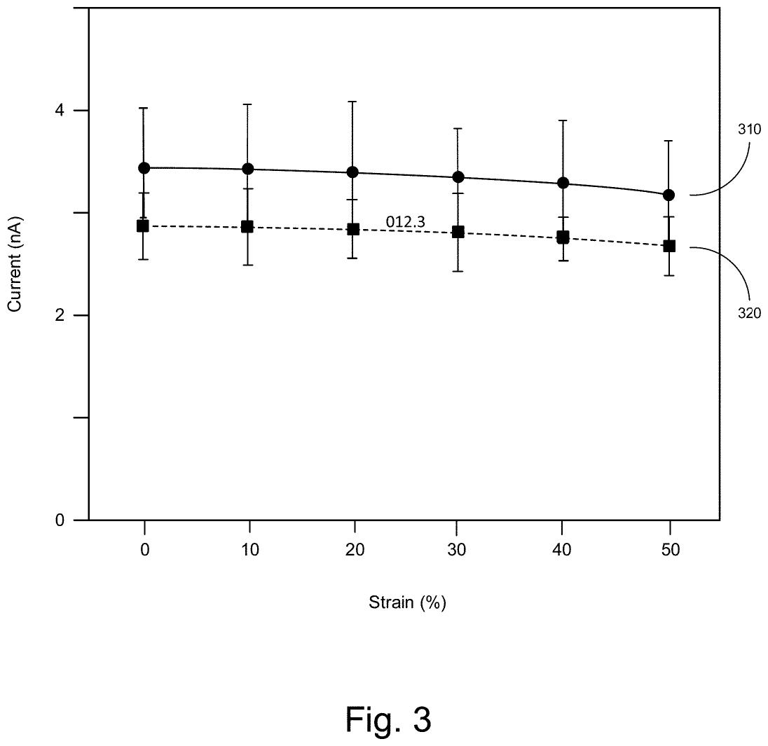

FIG. 3 illustrates a first example operating response of an implantable sensor device in accordance with present implementations. As illustrated by way of example in FIG. 3, an example operating response 300 can include a dopamine response 310 under variable strain, and a serotonin response 320 under variable strain.

Graphene nanofibers of the sensor strings 140A, 142A and 144A and sensor leads 250, 252 and 254 can maintain an interconnected 3D network after being transferred and embedded into substrate 110 and 210, respectively. When stretched to 100% strain, the graphene nanofiber network can align along the stretching direction, which can help to maintain the conductive pathways. Stretchability of the of the sensor strings 140A, 142A and 144A and sensor leads 250, 252 and 254 can also depend on power level of the laser applied during fabrication. Generally, a higher laser power can correspond to an increase of graphene loading in the elastomer as a thicker layer of polymer becomes carbonized. A graphene-elastomer composite can thus exhibit a much higher strain to failure, on the order of 1700% strain to failure or greater, compared to an SEBS elastomer alone. Graphene can increase Young's modulus when added in epoxy. Correspondingly, the graphene-in-elastomer composite can demonstrate a slightly decreased Young's modulus compared with that of the neat elastomer. In addition, the sheet resistance of the composite under 50% strain can stable with a change less than 200%, for products regardless of laser power applied during fabrication As one example, composite thickness of 50 μm, 90 μm, and 150 μm can respectively correspond to laser power of 6 W, 9 W, and 12 W.

In accordance with present implementations, graphene electrodes decorated with Fe₃O₄ nanoparticles have selectivity and sensitivity sufficient to distinguish DA and 5-HT, compared to conventional materials which cannot distinguish these biochemicals in this context. Stretching the device along different directions has minimum effect on the electrode impedance at less than 1000 Hz, revealed by a stable baseline under strain. Accordingly, both the dopamine response 310 and the serotonin response 320 demonstrate stable current response between 2 nA and 4 nA at strain levels at least between 0% and 50%. Thus, present implementations can demonstrate stable oxidation current peaks as a result of electrodes and sensors fabricated in graphene with Fe₃O₄ nanoparticles, under different strains. Present implementations also allow simultaneous sensing of 5-HT and other catecholamines (norepinephrine and epinephrine) with high selectivity.

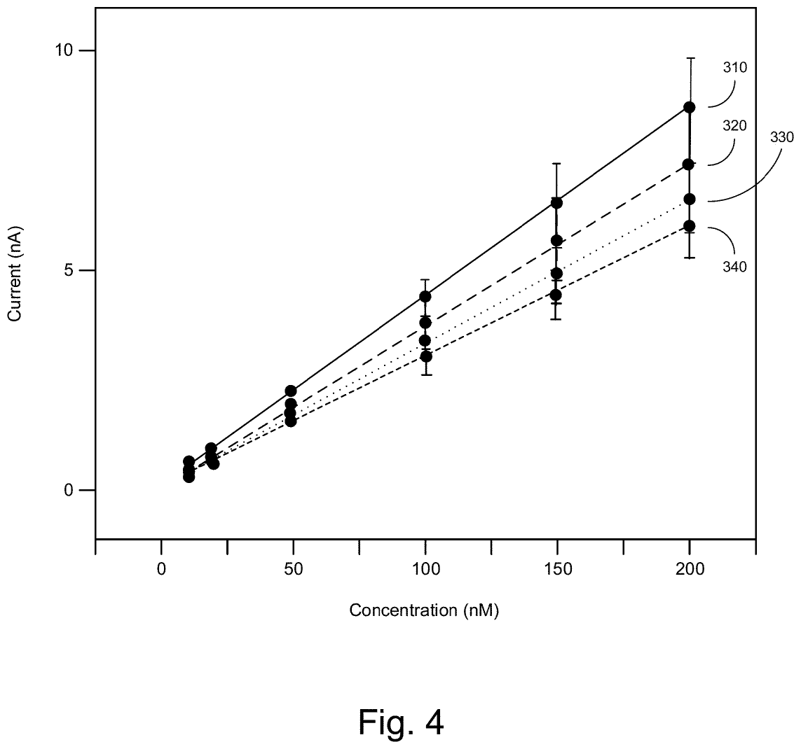

FIG. 4 illustrates a second example operating response of an implantable sensor device in accordance with present implementations. As illustrated by way of example in FIG. 4, an example operating response 400 can include a dopamine response 410 under variable concentration, a serotonin response 420 under variable concentration, a norepinephrine response 430 under variable concentration, and an epinephrine response 440 under variable concentration.

The dopamine response 410 can include a current response that increases monotonically and linearly between 0 nA and 10 nA in the presence of concentrations from 0 nM to 200 nM. In addition, cyclic voltammetry of the dopamine response 410 can demonstrate a current response trough at −5 nA and a current peak above 5 nA at concentrations between 10 nM and 200 nM. The serotonin response 420 can include a current response that increases monotonically and linearly between 0 nA and 10 nA in the presence of concentrations from 0 nM to 200 nM. In addition, cyclic voltammetry of the serotonin response 420 can demonstrate a current response trough at −5 nA and a current peak above 5 nA at concentrations between 10 nM and 200 nM. The norepinephrine response 430 can include a current response that increases monotonically and linearly between 0 nA and 10 nA in the presence of concentrations from 0 nM to 200 nM. In addition, cyclic voltammetry of the norepinephrine response 430 can demonstrate a current response trough below −5 nA and a current peak above 5 nA at concentrations between 10 nM and 200 nM. The epinephrine response 440 can include a current response that increases monotonically and linearly between 0 nA and 10 nA in the presence of concentrations from 0 nM to 200 nM. In addition, cyclic voltammetry of the serotonin response 420 can demonstrate a current response trough below −5 nA and a current peak above 5 nA at concentrations between 10 nM and 200 nM. Thus, present implementations can advantageously detect the presence and amount of multiple biochemicals including dopamine, serotonin, norepinephrine, and epinephrine within a predictable common current response window for at least all of these example biochemicals.

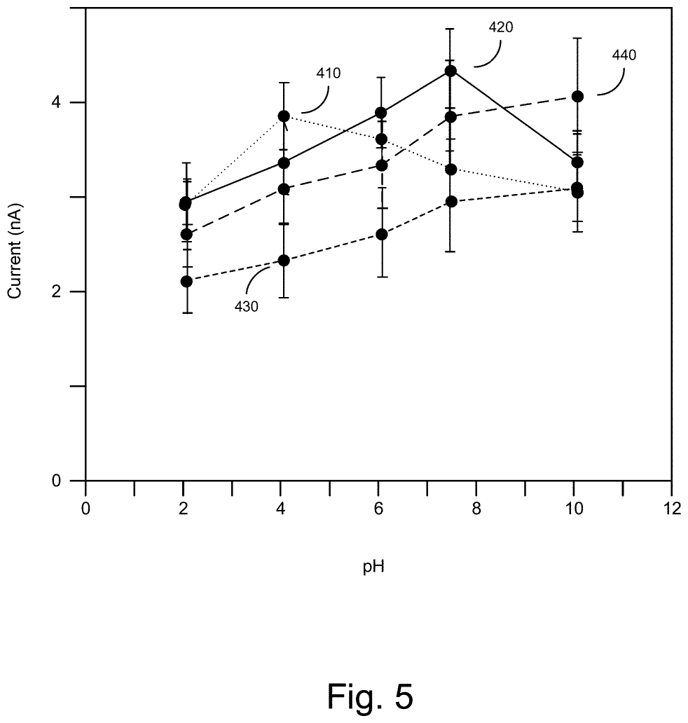

FIG. 5 illustrates a third example operating response of an implantable sensor device in accordance with present implementations. As illustrated by way of example in FIG. 5, an example operating response 500 can include a dopamine response 510 under variable pH, a serotonin response 520 under variable pH, a norepinephrine response 530 under variable pH, and an epinephrine response 540 under variable pH.

Transition metal nanoparticles, including Fe₃O₄ or NiO nanoparticles can promote molecular absorption and electron transfer to catalytically enhance selectivity and sensitivity for monoamine sensing. Thus, present implementations can include metalloporphyrin, 5,10,15,20-tetrakis-(4′-aminophenyl) iron (III) porphyrin chloride or 5,10,15,20-tetrakis-(4′-aminophenyl) nickel (II) porphyrin, into polyamic acid polymer as a precursor to produce nanoparticle-modified graphene networks through the laser carbonization process. The dopamine response 510 can include a current response that includes a current peak of 4 nA at pH 4, and a current minimum between 2 nA and 3 nA between pH 2 and pH 10. The serotonin response 520 can include a current response that includes a current peak between 4 nA and 5 nA at pH 8, and a current minimum between 2 nA and 3 nA between pH 2 and pH 10. The norepinephrine response 530 can include a current response that increases monotonically from a current minimum of 2 nA at pH 2 to a current peak between 2 nA and 3 nA at pH 10. The epinephrine response 540 can include a current response that increases monotonically from a current minimum between 2 nA and 3 nA at pH 2 to a current peak between 3 nA and 4 nA at pH 10. Each of the responses 510, 520, 530 and 540 can be linear response curves ranging from 10 nM to 200 nM concentration. Present implementations thus advantageously demonstrate stable current response across different pH.

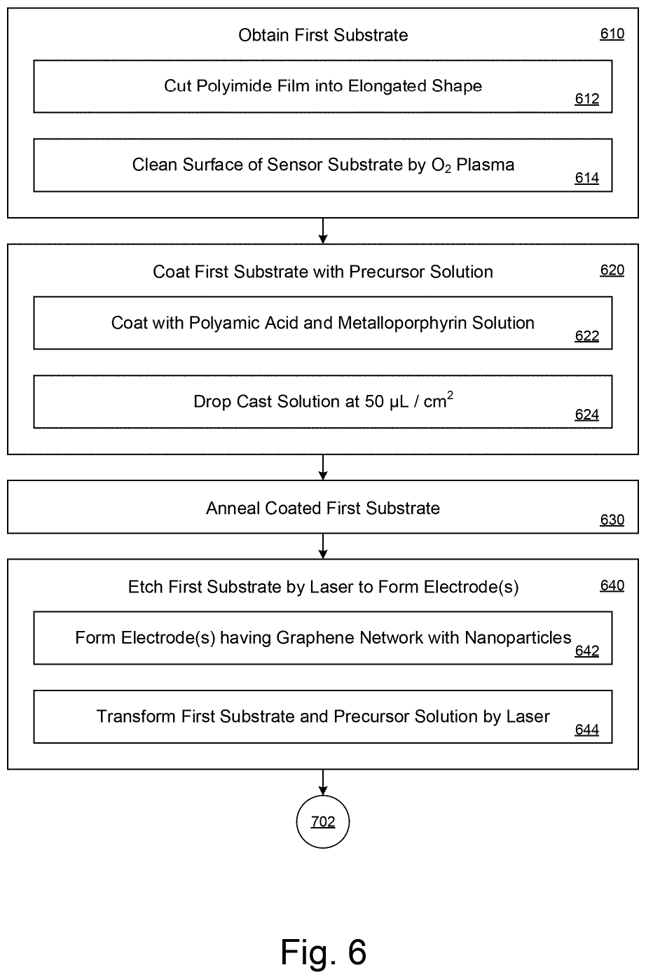

FIG. 6 illustrates a method of manufacturing an implantable device including a flexible biochemical sensor, in accordance with present implementations. At least one of the sensor devices 100A-C and 200A-C can be manufactured in accordance with method 600 according to present implementations. The method 600 can begin at step 610. The NeuroString fabrication process is based on direct laser carbonization of above polyimide polymer precursor (containing metalloporphyrin) into conductive nanoporous graphene networks decorated with nanoparticles

At step 610, the method can obtain a first substrate. The first substrate can include a polyimide film 12″ wide, 0.0050″ thick, and 12″ long. Step 610 can include at least one of steps 612 and 614. At step 612, the method can cut polyimide film into an elongated shape. The polyimide film can be first cut into a rectangle shape with a dimension of 3″×2″. At step 614, the method can clean the surface of a sensor substrate by an oxygen (e.g., O₂) plasma. The O₂ plasma can be applied at 150 W, 200 mTorr for 2 min. The method 600 then continues to step 620.

At step 620, the method can coat a first substrate with a precursor solution. Step 620 can include at least one of steps 622 and 624. At step 622, the method can coat the first substrate with a solution including one or more of polyamic acid and metalloporhyrin. At step 624, the method can drop cast the solution at an amount of 50 μL/cm². The coated first substrate can be left to settle in air for 1 hour to form a uniform liquid film with flat surface. Then, the coated first substrate can be loaded on a hotplate at 100° C. for 1 hour to remove all solvent. The method 600 then continues to step 630.

At step 630, the method can anneal the coated first substrate. The coated first substrate can then be annealed at 150° C. for 5 min, and annealed again at 250° C. for 1 hour. The method 600 then continues to step 640.

At step 640, the method can etch a first substrate by a laser to form one or more electrodes. Step 640 can include at least one of steps 642 and 644. At step 642, the method can form electrodes having a graphene network with nanoparticles. The method can form laser-induced graphene with 0.34 nm d-spacing. At step 644, the method can transform the first substrate and the precursor solution by a laser. A laser power of 6 W can be used with a raster speed of 25%. The desired electrochemical properties, combined with the rapid laser patterning and the ease of transfer process enabled by SEBS, result in a versatile materials platform with high stretchability and rapid fabrication of arbitrary patterns. The method 600 then continues to step 702.

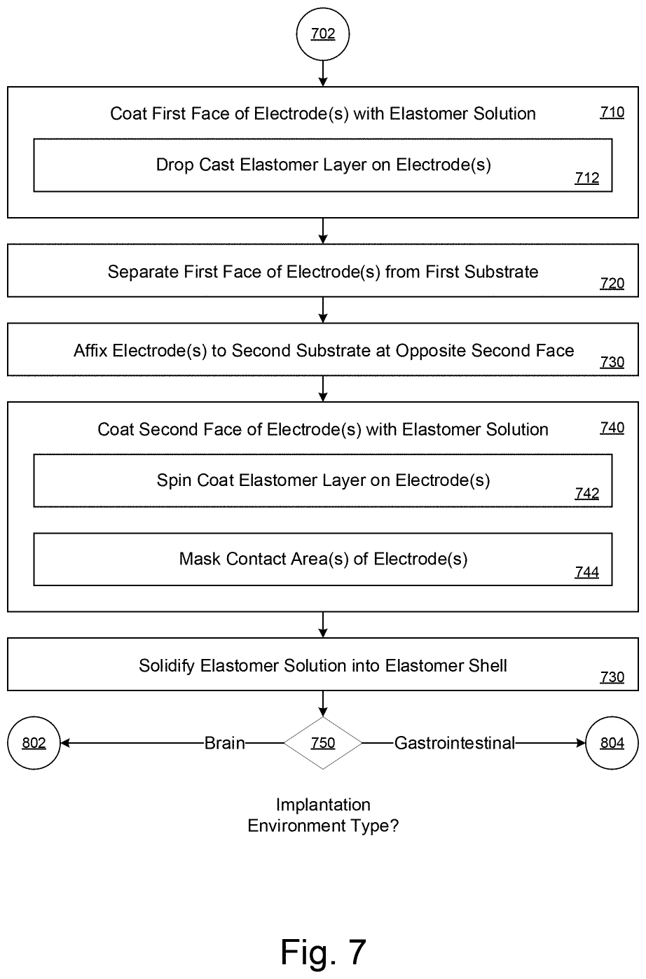

FIG. 7 illustrates a method of manufacturing an implantable device including a flexible biochemical sensor, further to the example method of FIG. 6. At least one of the sensor devices 100A-C and 200A-C can be manufactured in accordance with method 700 according to present implementations. The method 700 can begin at step 702. The method 700 then continues to step 710.

At step 710, the method can coat a first face of the electrodes with an elastomer solution. The elastomer solution can include poly(Styrene-co-Ethylene-co-Butylene-Styrene) (SEBS) in toluene at 0.1 g/mL. Step 710 can include step 712. At step 712, the method can drop cast an elastomer layer on the electrodes. The elastomer layer can be drop-casted on the graphene networks at 100 μL/cm² and then placed in a desiccator with a house vacuum for 5 min to remove all gas bubbles. The method 700 then continues to step 720. At step 720, the method can separate a first face of the electrodes from the first substrate. After allowing toluene solvent to evaporate, the graphene/SEBS composite can be removed from the first substrate. The composite can be removed from the first substrate by peeling the composite from the polyimide. The method 700 then continues to step 730. At step 730, the method can affix the electrodes to a second substrate at a second face of the electrodes opposite to the first face. The composite can be flipped to expose the graphene network and placed on another glass substrate. The method 700 then continues to step 740.

At step 740, the method can coat a second face of the electrodes with an elastomer solution. Step 740 can include at least one of steps 742 and 744. At step 742, the method can spin coat an elastomer layer on the electrodes. The elastomer solution of step 740 can correspond at least partially to the elastomer solution of step 710. The elastomer solution can be spin-coated on top at 1,000 r.p.m., to form a stretchable encapsulation layer. At step 744, the method can mask one or more contact areas of the electrodes. The contact areas can correspond to portion of the electrode surfaces contactable with electrical wires or connectors, for example. The method 700 then continues to step 750. At step 750, the method can solidify an elastomer solution into an elastomer shell. The method 700 then continues to step 760.

At step 760, the method can continue in accordance with an implantation environment type. In accordance with an implantation environment corresponding to a brain environment, the method 700 continues to step 802. Alternatively, in accordance with an implantation environment corresponding to a gastrointestinal environment, the method 700 continues to step 804.

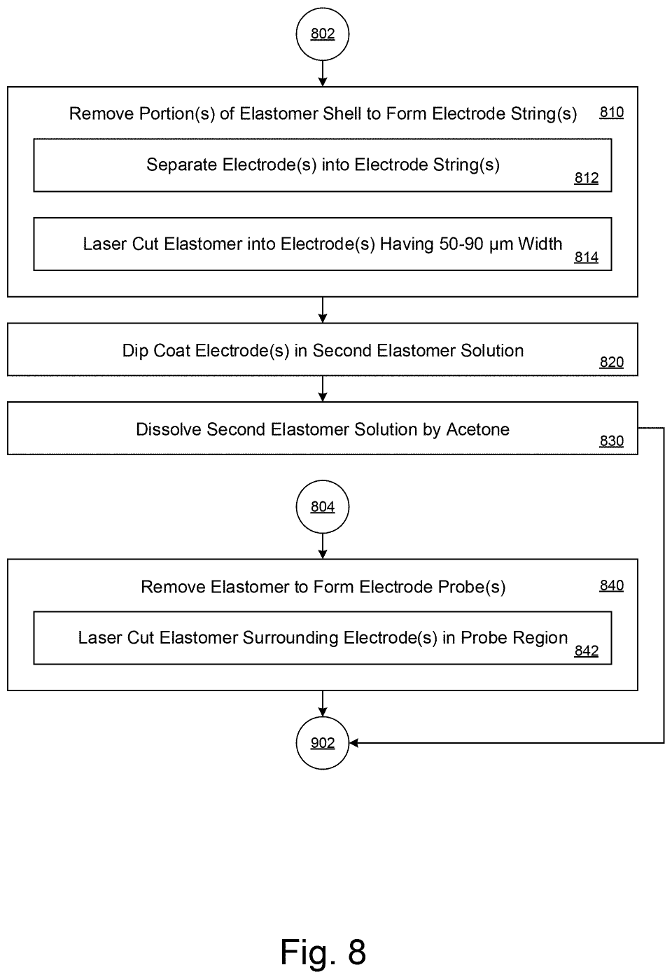

FIG. 8 illustrates a method of manufacturing a brain-implantable or gastrointestinal-implantable device including a flexible biochemical sensor, further to the example method of FIG. 7. At least one of the sensor devices 100A-C and 200A-C can be manufactured in accordance with method 800 according to present implementations. The method 800 can begin at step 802. The method 800 then continues to step 810.

At step 810, the method can remove one or more portions of the elastomer shell to form one or more electrode strings. After removal of the tape, the film was attached to a glass slide and laser cutting was used to define the individual separated electrodes Step 810 can include at least one of steps 812 and 814. At step 812, the method can separate one or more of the electrodes into one or more corresponding electrode strings. At step 814, the method can cut the elastomer by a laser into one or more electrode strings having widths of 50-90 μm. A laser power of 30 W can be used to excess elastomer between electrode strings and to form small individual electrodes having particular widths. The method 800 then continues to step 820.

At step 820, the method can dip coat one or more electrodes in a second elastomer solution. The electrodes can be dip-coated with a second SEBS solution to fully encapsulate the sidewalls of graphene electrodes. The second SEBS solution can include 0.1 g/mL in acetone. The method 800 then continues to step 830. At step 830, the method can dissolve the second elastomer solution by acetone. Acetone can be used to dissolve the second SEBS in order prevent dissolution of the formed electrodes and substrate. The method 800 then continues to step 902.

At step 804, the method can continue to step 840. At step 840, the method can remove one or more portions of the elastomer shell to form at least one electrode probe. Step 840 can include step 842. At step 842, the method can laser cut elastomer surrounding the electrodes in a probe region associated with the electrodes. A laser cutting step can shape the probes by one or more cuts through the elastomer in a direction of cutting. A CO₂ laser with a 30 W power can be used to remove excess portions of elastomer. The method can cut a sensor probe region and fabricate electrodes, or both, having a width of 1 cm or less. The width can depend on the location of probe insertion. For example, larger probes can be used for intestine while smaller probes are advantageous for inserting into brain. In the instance of elastomer probe formation, individual electrodes can have a width of approximately 120-220 μm. Smaller probes can be made as needed. The method 800 then continues to step 902.

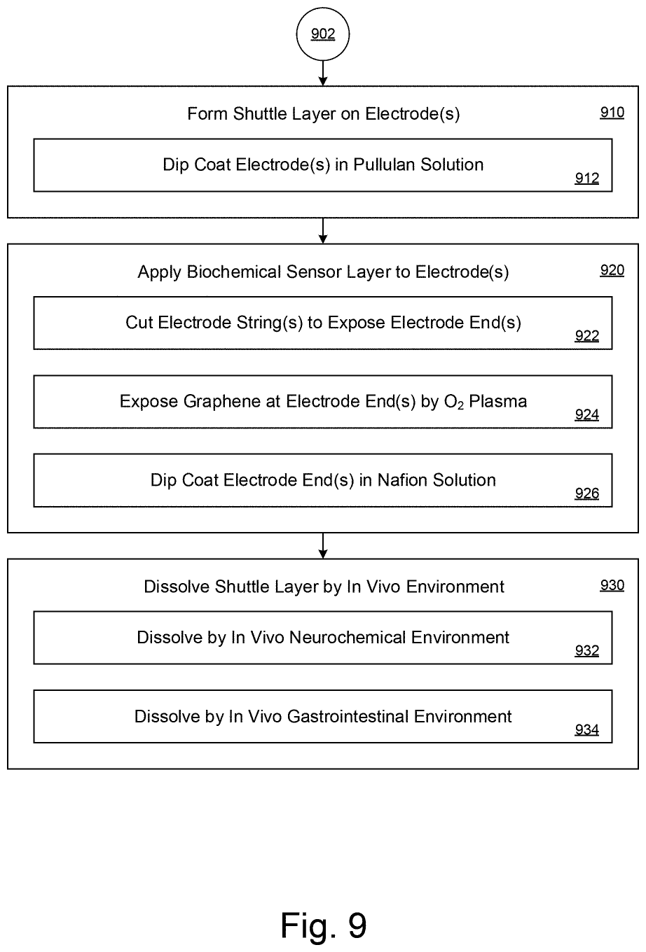

FIG. 9 illustrates a method of manufacturing an implantable device including a flexible biochemical sensor, further to the example method of FIG. 8. At least one of the sensor devices 100A-C and 200A-C can be manufactured in accordance with method 900 according to present implementations. The method 900 can begin at step 902. The method 900 then continues to step 910.

At step 910, the method can form at least one shuttle layer on the electrodes. Step 910 can include step 912. At step 912, the method can dip coat the electrodes in a solution containing a biocompatible solid that dissolves in water, such as a pullulan solution. With respect to a CNS sensor device, a pullulan solution can have a concentration of 0.3 g/mL. This concentration can be adjusted depending on the bending stiffness required for probe insertion. A thicker coating will result in a higher bending stiffness. With respect to a GI sensor device, a pullulan solution can have a concentration of 0.1 g/mL. The method 900 then continues to step 920.

At step 920, the method can apply at least one biochemical sensor layer to the electrodes. The biochemical sensor layer can reduce a biofouling effect from interferents present with one or more target analytes as discussed in FIGS. 3-5. Step 920 can include at least one of steps 922, 924 and 926. At step 922, the method can cut one or more of the electrode strings to expose one or more electrode ends of the cut electrode strings. At step 924, the method can expose graphene at electrode ends by oxygen (e.g., O₂) plasma. To fully expose the graphene, the cross-sectional surfaces of electrodes can be oxygen plasma-treated at 150 W, 200 mTorr for 2 min. At step 926, the method can dip coat one or more of the electrode ends in Nafion. To avoid any interference of the ascorbic acid and biofouling from the biological fluids, the tip of the electrodes can be dipped in a Nafion solution to form a Nafion coating before use. The Nafion solution can be 0.5% in water/ethanol. The method 900 then continues to step 930.

At step 930, the method can dissolve the shuttle layer by, for example, contact with or exposure to the in vivo environment. Step 930 can include at least one of steps 932 and 934. At step 932, the method can dissolve the shuttle layer in an in vivo neurochemical environment. Once the sensor device 100C is implanted in the brain tissue, the sensor device 100C can be released after pullulan dissolves away to expose the soft implanted electrode. At step 934, the method can dissolve the shuttle layer in an in vivo gastrointestinal environment. Once the sensor device 200C is implanted in the GI tissue, the sensor device 200C can be released after pullulan dissolves away to expose the soft implanted electrode. In some implementations, the method 900 ends at step 930. It is to be understood that the in vivo environment is not limited to a neurochemical in vivo environment or an in vivo gastrointestinal environment. Implantable sensor devices in accordance with present implementations can sense any chemical to which the receptor is modified on the surface of the electrode. Thus, implantable electrodes in accordance with present implementations can sense at least presence and magnitude of at least pH, glucose, pressure, temperature, and strain. Present implementations can also conduct electrophysiological signal sensing and electrical stimulation.

The herein described subject matter sometimes illustrates different components contained within, or connected with, different other components. It is to be understood that such depicted architectures are illustrative, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It will be further understood by those within the art that if a specific number of an introduced claim recitation is intended, such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general, such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Further, unless otherwise noted, the use of the words “approximate,” “about,” “around,” “substantially,” etc., mean plus or minus ten percent.

The foregoing description of illustrative implementations has been presented for purposes of illustration and of description. It is not intended to be exhaustive or limiting with respect to the precise form disclosed, and modifications and variations are possible in light of the above teachings or may be acquired from practice of the disclosed implementations. It is intended that the scope of the invention be defined by the claims appended hereto and their equivalents.

What is claimed is:

1. A method of forming an implantable biochemical sensor, the method comprising:
coating a first substrate with a first solution;
etching, by a laser, the first substrate to form one or more electrodes in the first substrate;
coating a first face of the electrodes with an elastomer solution;
coating a second face of the electrodes with the elastomer solution;
solidifying the elastomer coating into an elastomer shell at least partially surrounding the electrodes; and
removing at least a portion of the elastomer shell to form implantable electrodes by exposing a portion of the electrodes.

2. The method of claim 1, wherein the removing further comprises:
removing the portion of the elastomer shell to form implantable electrode strings.

3. The method of claim 2, wherein the electrode strings each have a width less than 100 μm.

4. The method of claim 1, wherein the removing further comprises:
removing the portion of the elastomer shell to form an implantable electrode probe.

5. The method of claim 1, further comprising:
forming a shuttle layer on the implantable electrodes.

6. The method of claim 5, further comprising:
dissolving at least a portion of the shuttle layer by contact with an in vivo neurochemical environment.

7. The method of claim 5, further comprising:
dissolving at least a portion of the shuttle layer by contact with an in vivo gastrointestinal environment.

8. The method of claim 1, further comprising:
applying a biochemical sensor layer to corresponding implantable ends of the electrodes.

9. The method of claim 1, wherein the first solution includes one or more of polyamic acid and metalloporhyrin.

10. The method of claim 1, wherein the electrodes comprise a graphene network.

11. The method of claim 10, the etching further comprising:
transforming, by the laser, the first substrate and the first coating into the graphene network.

12. The method of claim 11, the etching further comprising:
generating, by the laser, nanoparticles within the graphene network.

13. An implantable biochemical sensor device, the device comprising:
a first biochemical sensor comprising a first portion of a graphene network, and having an elongated structure with a first input end and a first sensor end distal to the first input end; and
a second biochemical sensor comprising a second portion of the graphene network, and having the elongated structure with a second input end and a second sensor end distal to the second input end.

14. The device of claim 13, wherein the first sensor end and the second sensor end each comprise implantable electrode strings each having a width less than less than 100 μm.

15. The device of claim 13, wherein the first sensor end and the second sensor end are electrically responsive to one or more of dopamine, serotonin, norepinephrine, and epinephrine.

16. The device of claim 13, further comprising:
an elastomer coating at least partially surrounding the first biochemical sensor and the second biochemical sensor.

17. The device of claim 13, further comprising:
an elastomer coating individually surrounding each of the first sensor end and the second sensor end.

18. The device of claim 13, further comprising:
an optical fiber operatively coupled with at least one of the first biochemical sensor and the second biochemical sensor, the optical fiber operable to transmit light to perform optogenetic stimulation, optical biosensing, or light therapy within an in vivo environment.

19. The device of claim 13, wherein the graphene network is operable to transmit an electrical stimulation signal and receive an electrical signal from an in vivo environment.

20. The device of claim 13, wherein at least a portion of the graphene includes at least one of a biochemical receptor and a chemical receptor.
